(19) United States
(12) Patent Application Publication
(10) Pub. No.: US 2024/0265545 A1
(43) Pub. Date: Aug. 8, 2024

(54) CORRELATING MULTI-MODAL MEDICAL IMAGES

(71) Applicant: Roche Molecular Systems, Inc., Pleasanton, CA (US)

(72) Inventors: Dennive MALIKSI, Pleasanton, CA (US); Ellen SUZUE, Pleasanton, CA (US); Mateusz SZEWCZYK, Pleasanton, CA (US); Guillaume BOUSQUET, Pleasanton, CA (US); Christophe CHEFD'HOTEL, Pleasanton, CA (US)

(21) Appl. No.: 18/603,651

(22) Filed: Mar. 13, 2024

Related U.S. Application Data
(63) Continuation of application No. PCT/US2022/036952, filed on Jul. 13, 2022.
(60) Provisional application No. 63/244,756, filed on Sep. 16, 2021.

(51) Int. Cl. G06T 7/00; A61B 6/03; A61B 6/46; G06T 7/73; G16H 30/40; G16H 40/63

(52) U.S. Cl. CPC G06T 7/0014; A61B 6/037; A61B 6/463; A61B 6/465; G06T 7/73; G16H 30/40; G16H 40/63; G06T 2200/24; G06T 2207/10104; G06T 2207/20081; G06T 2207/20084; G06T 2207/20104; G06T 2207/30096

(57) ABSTRACT

In some embodiments, methods, systems, software and uses are provided for synchronizing medical images displays. An input identifying a region of interest in a first medical image is received. A second region of interest in a second medical image is determined based the first region. The first medical image and a first indication of the first region are displayed in a first viewport of a GUI. The second medical image and a second indication of the second region are displayed in a second viewport of the GUI. A display adjustment input is received to adjust the displaying of one of the first or second region of interest. Based on the display adjustment input and the correspondence information, an adjustment of the displaying of the first region of interest in the first viewport and an adjustment of the displaying of the second region of interest in the second viewport is implemented.

### CROSS-REFERENCE TO RELATED APPLICATIONS

This application is a bypass continuation of International Application No. PCT/US2022/036952, filed on Jul. 13, 2022, which claims the benefit of and the priority to U.S. Application No. 63/244,756, filed on Sep. 16, 2021, which are hereby incorporated by reference in their entireties for all purposes.

### BACKGROUND

Medical images generally refer to images that are taken for clinical analysis and/or intervention. Multi-modal medical images can be obtained from multiple imaging/processing modalities. For example, various types of radiology images can be taken to reveal the internal structures of subjects (e.g., patients), or to detect certain cells of interest (e.g., cancer cells), without performing invasive procedures, such as positron emission tomography (PET), X-ray radiography, magnetic resonance imaging, ultrasound, single-photon emission computed tomography (SPECT), etc. As another example, tissue specimens can be removed from the subjects and sliced into specimen slides. The specimen slides can be further processed (e.g., Hematoxylin and Eosin (H&E) staining, Immunohistochemistry (IHC) staining, fluorescent tagging, etc.) and/or illuminated (e.g., with fluorescent illumination, bright-field (visible light) illumination, etc.), and digital pathology images can be taken of the processed/illuminated slides to provide histology information of cells in the specimen. Radiology images taken using different types of radiology techniques can also be regarded as multi-modal medical images, as can pathology images taken from slides processed with different staining agents/techniques to reveal different types of cell/tissue structures, and so on.

Given that these multi-modal medical images provide different modalities of information, typically these images are displayed as two discrete pieces of medical data to be analyzed by different specialists. For example, radiology images are to be reviewed and analyzed by radiologists, whereas digital pathology images are to be reviewed and analyzed by pathologists. Currently, medical information systems (e.g., a digital imaging and communications in medicine (DICOM) system) that provides access to these images typically do not allow a user to efficiently determine a correspondence between two medical images of different modalities such as, for example, identifying which region in one medical image corresponds to another region in another medical image.

Moreover, current medical information systems also do not provide easy and intuitive ways to store and access the information indicating the corresponding regions between two medical images. In a case where multi-modal medical images capture different extent of the subject's body, these images tend to have different resolutions and represent different scales. Without information indicating corresponding regions between two images, it becomes challenging for the user to navigate in each of the two images to locate and to access the corresponding regions of interest.

Therefore, there is a need to provide a system that can automatically correlate multi-modal medical images by determining correspondence portions between the images, and/or allow a user to easily mark and store information indicating corresponding regions between multi-modal medical images.

### BRIEF SUMMARY

Disclosed herein are techniques to correlate multi-modal medical images and to provide access to the correlation results. The multi-modal medical images include a first medical image and a second medical image obtained from different imaging/processing modalities. In some examples, the first medical image can include a digital radiology image, whereas the second medical image can include a digital pathology image. In some examples, both the first medical image and the second medical image can include digital radiology images or digital pathology images obtained using different techniques to reveal different information.

In some examples, the techniques include accessing, from one or more databases, the first medical image and the second medical image, and receiving, via a graphical user interface (GUI) and from a user, a selection input corresponding to selection of a first region of interest in the first medical image. The techniques further include determining a second region of interest in the second medical image based on the first region of interest and the second region of interest corresponding to the same tissue. The techniques further include determining, based on the information that the first region of interest is associated with in the second region of interest, and storing correspondence information indicating a first location of the first region of interest, a second location of the second region of interest, and the association between the first region of interest and the second region of interest. The techniques further include displaying, in the GUI, the first medical image, a first indication of the first region of interest, the second medical image, and the second region of interest. The techniques further include receiving a display adjustment input via the GUI to adjust the displaying of one of the first region of interest or the second region of interest in the GUI and, synchronizing, based on the display adjustment input and the correspondence information, between an adjustment of the display of the first region of interest and an adjustment of the display of the second region of interest in the GUI.

These and other examples of the present disclosure are described in detail below. For example, other embodiments are directed to systems, devices, and computer readable media associated with methods described herein.

A better understanding of the nature and advantages of the disclosed techniques may be gained with reference to the following detailed description and the accompanying drawings.

### BRIEF DESCRIPTION OF THE DRAWINGS

The detailed description is set forth with reference to the accompanying figures.

FIG. 1A and FIG. 1B illustrate examples of multi-modal medical images.

FIG. 2A and FIG. 2B illustrate an example of a multi-modal medical images correlating system, according to certain aspects of the present disclosure.

FIG. 3A, FIG. 3B, and FIG. 3C illustrate examples of correspondence information generated by the multi-modal medical images correlating system of FIG. 2A and FIG. 2B, according to certain aspects of the present disclosure.

FIG. 4A, FIG. 4B, and FIG. 4C illustrate examples of internal components of the multi-modal medical images correlating system of FIG. 2A and FIG. 2B, according to certain aspects of this disclosure.

FIG. 5A, FIG. 5B, and FIG. 5C illustrate examples of display operations supported by the example multi-modal medical images correlating system of FIG. 2A and FIG. 2B, according to certain aspects of the present disclosure.

FIG. 6 illustrates examples of internal components of the multi-modal medical images correlating system of FIG. 2A and FIG. 2B, according to certain aspects of this disclosure.

FIG. 7A, FIG. 7B, FIG. 7C, FIG. 7D, FIG. 7E, and FIG. 7F illustrate examples of a graphical user interface (GUI) provided by the multi-modal medical images correlating system of FIG. 2A and FIG. 2B, according to certain aspects of this disclosure.

FIG. 8 illustrates a method of displaying multi-modal medical images, according to certain aspects of this disclosure.

FIG. 9 illustrates an example computer system that may be utilized to implement techniques disclosed herein.

### DETAILED DESCRIPTION

Disclosed herein are techniques for correlating multi-modal medical images. The multi-modal medical images include a first medical image and a second medical image of a subject obtained from different imaging/processing modalities to support a particular clinical analysis for the subject, such as a cancer diagnosis. In some examples, the first medical image can include a digital radiology image, whereas the second medical image can include a digital pathology image. In some examples, both the first medical image and the second medical image can include digital radiology images or digital pathology images but obtained using different techniques (e.g., different types of staining, different types of illuminations, etc.) to reveal different information.

In some examples, the techniques can be implemented by an inter-modality medical images correlating system. The system can access the first medical image and the second medical image from one or more data sources, such as databases, a user device, etc. The first medical image can include a digital radiology image, such as a PET image that reveals a distribution of radioactive levels within the subject's body, whereas the second medical image can include a digital pathology image of the subject's tissue. In some example, the distribution of radioactive levels shown in the PET image can identify potential tumor locations in the subject's body, whereas digital pathology image can include an image of a sample (e.g., a tissue specimen) collected from the subject that has been stained (e.g., H&E staining, IHC staining, fluorescent tagging, etc.) and/or illuminated (e.g., fluorescent illumination, bright-field illumination, etc.) to reveal suspected tumor cell. The databases may include, for example, an electronic medical record (EMR) system, a picture archiving and communication system (PACS), a Digital Pathology (DP) system, a laboratory information system (LIS), and a radiology information system (RIS).

The inter-modality medical images correlating system can further provide a GUI. In some examples, the system can receive a selection input via the GUI to select a first region of interest in the first medical image. Specifically, in some examples, the selection input can include a selection of one or more first image locations in the first medical image as one or more first landmark points. The first region of interest can encompass the first landmark points. The first region of interest can be of various geometric shapes, such as a triangular shape, a rectangular shape, a freeform shape, etc., which can be based on the number of first landmark points. For example, the first region of interest can correspond to a region having a radioactive level in a PET image, which can indicate the presence of a tumor that metabolizes a radiolabeled glucose tracer injected into the subject's body. In some examples, the selection input can also include a direct selection of the first region of interest by a user. In some examples, an image processing application of the multi-modal medical images correlating system can process the first medical image by comparing the radioactive level revealed in the PET image with a threshold. One or more candidate first regions of interest in the first image can be defined based on the comparison result. For example, the one or more candidate first regions of interest in the first image can be defined based on regions having radioactive level higher than the threshold. Multiple candidate first regions of interest may be identified in a case where there are multiple suspected tumor sites in the subject's body. The selection input can be received from the user to select one of the candidate first regions of interest as the first region of interest that corresponds to, for example, a tumor site at a particular location of the subject's body. Based on the selection input, the system can determine various information of the first region of interest including, for example, a first location (e.g., a center location) of the first region of interest, a shape of the first region of interest, a size of the first region of interest, etc.

The inter-modality medical images correlating system can also determine a second region of interest in the second medical image. The second region of interest can be determined based on, for example, determining the tissue (e.g., a tumor tissue) represented by the first region of interest, followed by identifying the second region of interest in the second medical image that corresponds to the same tissue (e.g., the same tumor tissue). In some examples, the determination can be based on receiving a second selection input from the user. The second selection input may include selection of one or more second image locations in the second medical image as one or more second landmark points, and the second region of interest can encompass the second landmark points. In some examples, the information can also be determined based on inputs from the user. For example, the GUI may provide a corresponding regions of interest input option to enter landmark points of a pair of corresponding regions of interest in the first medical image and in the second medical image. By receiving the first and second landmark points via the corresponding region of interest input option, the multi-modal medical images correlating system can determine the information indicating that the first region of interest and the second region of interest correspond to the same tissue. The system can also determine various information of the second region of interest including, for example, a second location (e.g., a center location) of the second region of interest, a shape of the second region of interest, a size of the second region of interest, etc., based on the landmark points in the second medical image.

In some examples, the second region of interest can also be determined by a machine learning model of the multi-modal medical images correlating system. The machine learning model can determine, for each pixel of the second medical image, a likelihood of the pixel belonging to the tissue, and classify that a pixel belongs to the tissue, and that the pixel is to be included in the second region of interest, if the likelihood exceeds a threshold. Based on the classification results, the multi-modal medical images correlating system can then determine the second region of interest in the second medical image to include pixels that are classified as part of the tissue. In some examples, the machine learning model can include a deep convolutional neural network (CNN) comprising multiple layers. The CNN can perform convolution operations between the second medical image and weight matrices representing features of the tissue to compute the likelihoods of the pixels belonging to the tissue, and to determine the pixels that are part of the second region of interest. The system can then determine various information of the second region of interest including, for example, a second location (e.g., a center location) of the second region of interest, a shape of the second region of interest, a size of the second region of interest, etc., based on pixels determined to be part of the second region of interest.

In some examples, after determining the first region of interest in the first medical image and the second region of interest in the second medical image, the inter-modality medical images correlating system can store correspondence information indicating one or more first locations of the first region of interest, one or more second locations of the second region of interest, and the correspondence/association between the first region of interest and the second region of interest. The first and second locations can include, for example, the boundary locations, center locations, etc., of the first region of interest and the second region of interest. In some examples, the correspondence information can include the pixel locations of the first landmarks and the second landmarks that can define, respectively, the first location of the first region of interest and the second location of the second region of interest. In some examples, the correspondence information may further include additional information, such as the locations of the boundaries of the first region of interest and the second region of interest, the file names of the first medical image and the second medical image, the type of tissue represented in the regions of interest, etc. In some examples, the correspondence information may include a data structure, such as a mapping table, that maps the first region of interest to the second region of interest. In some examples, the first medical image can be part of a 3D PET image, and the mapping table can include three dimensional coordinates of the first region of interest. The mapping table can also map the electronic file names of the first medical image to the second medical image if both medical images are 2D images. In a case where the first medical image is part of a 3D PET image that comprises multiple 2D PET images obtained at different longitudinal positions, the mapping table can map first regions of interest in multiple 2D PET images to second regions of interest in multiple second medical images. Such arrangements allow the multi-modal medical images correlating system to access the mapping table and the regions of interest information after accessing the first medical image and the second medical image.

After determining the first and second regions of interest, the multi-modal medical images correlating system can display the first medical image, a first indication of the first region of interest, the second medical image, and a second indication of the second region of interest in the GUI. Specifically, the GUI may include a first viewport to display the first medical image and the first indication, and a second viewport to display the second medical image and the second indication. The indication of a region of interest can be in various forms, such as the landmarks that define the region of interest, a geometric shape representing the region of interest, various forms of annotations, etc.

While displaying the first medical image and the second medical image, the multi-modal medical images correlating system can receive a display adjustment input via the GUI to adjust the displaying of one of the first region of interest or the second region of interest in one of the first viewport or the second view port. The display adjustment input can include, for example, a zoom-in/zoom-out input, a panning input, a rotation input, etc., to adjust the displaying of a region of interest in the viewport that receives the display adjustment input. The multi-modal medical images correlating system can also synchronize the adjustment of display in both viewports such that both viewports can display the same region indicated by the same set of coordinates. The multi-modal medical images correlating system can perform the synchronization based on the display adjustment input and the correspondence information. As a result of the synchronization, various settings of the display, such as a degree of magnification, the portion of the region of interest selected for display, a viewpoint of the region of interest, etc., are applied to both viewports, such that both viewports can display the same region indicated by the same set of coordinates in the first and second medical images.

For example, in a case where a zoom-in input is received at the first viewport to zoom into the first region of interest in the first viewport, the multi-modal medical images correlating system can compute a degree of magnification based on the zoom-in input, and magnify the first region of interest in the first viewport by the degree of magnification. In addition, the multi-modal medical images correlating system can also identify the second region of interest at the second location of the second medical image (based on the correspondence information), magnify the second region of interest by the same degree of magnification in the second viewport so that the first region of interest and the second region of interest are displayed to the same scale.

As another example, a panning input is received at the first viewport to pan to a selected portion of the first region of interest, and the multi-modal medical images correlating system can display the selected portion of the first region of interest. In addition, based on a mapping between first location of the first region of interest and the second location of the second region of interest, the multi-modal medical images correlating system can determine the corresponding portion of the second region of interest, and display the corresponding portion of the second region of interest in the second viewport.

In addition to synchronizing the displaying of the first region of interest and the second region of interest in their respective viewports, the multi-modal medical images correlating system can also support other types of display and analytics operations based on combining pathology features and radiology image features to support a clinical diagnosis, such as identification of cancer cells. For example, the multi-modal medical images correlating system can include a third viewport to display both the first region of interest on the second region of interest to the same scale, and overlay the first region of interest over the second region of interest, or vice versa. The overlaying region of interest can be displayed in a semi-transparent form. Such arrangements can support visual comparison between the first region of interest and the second region of interest. For example, the first region of interest may represent part of a body having an elevated radioactive level (from the radiolabeled glucose tracer), which can indicate the presence of a tumor. The second region of interest may reveal the actual tumor cells. A visual comparison between the two regions of interest can confirm the presence of a tumor, and/or verify that a prior cancer surgery has removed a cancerous tissue other than a healthy tissue. In some examples, the multi-modal medical images correlating system can include an image processing module to analyze the second region of interest (e.g., based on analyzing stain patterns) to detect cell structures that are indicative of tumor cells. A comparison between the locations of the tumor cells in the second region of interest and the elevated radioactive level in the first region of interest can also confirm the presence of a tumor.

The disclosed techniques can facilitate access and detection of corresponding regions between multi-modal medical images, such as between a radiology image and a pathology image, to facilitate a clinical analysis. Specifically, as described above, in a case where multi-modal medical images capture different extent of the subject's body, these images tend to have different resolutions and represent different scales. As the multi-modal medical images correlating system enables storage of the correspondence information that maps between regions of interest of the multi-modal medical images, and links the correspondence information to the electronic files of the multi-modal medical images, the system can facilitate a user's access to the regions of interest in the multi-modal images. Moreover, by synchronizing the display of the two corresponding regions of interest, the system allows a user to navigate through two corresponding regions of interest simultaneously in two medical images that have different resolutions/scales. In addition, some examples of the system can support automatic detection of first and second regions of interest in the multi-modal medical images, and the correspondence between the first and second regions of interest, which can further facilitate detection of regions of interest in the medical images despite the images having different scales/resolutions.

As described above, by providing the capability to correlate between regions of interest between multi-modal images, additional clinical diagnosis, such as confirmation of tumor cells, confirmation of surgical procedures, etc., can be performed. As a result the efficiency of analyzing and correlating between the multi-modal medical images by the physicians can be improved, which can also improve the quality of care provided to the subjects.

### I. Example Multi-Modal Medical Images

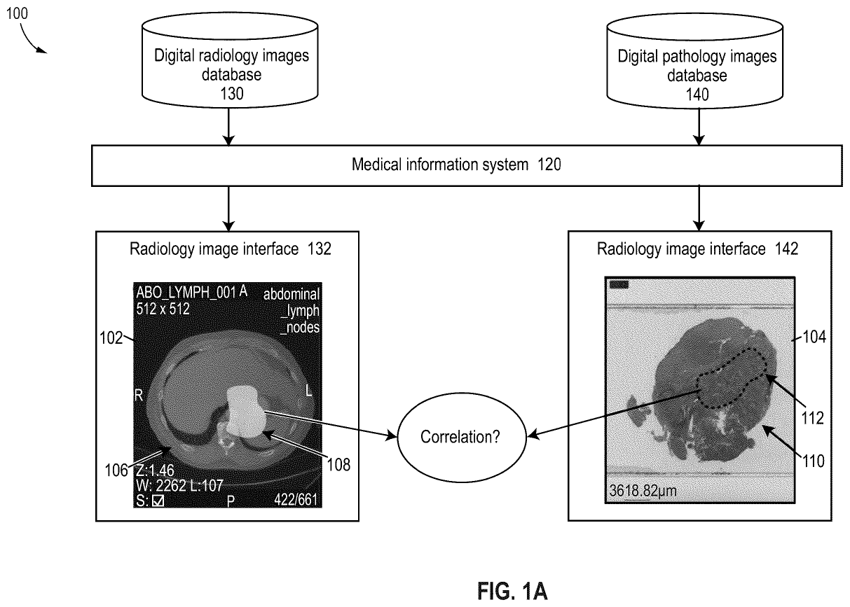

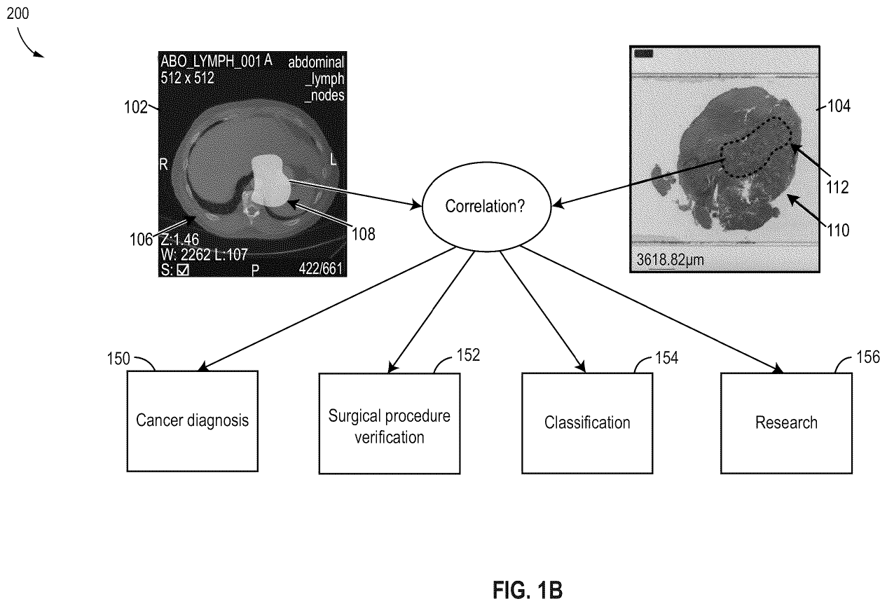

FIG. 1A and FIG. 1B illustrate examples of multi-modal medical images and how they may be used by physicians. As shown in FIG. 1A, two medical images of different modalities, including a first medical image 102 and a second medical image 104, can be displayed to physicians. First medical image 102 and second medical image 104 can be acquired from different imaging/processing modalities. For example, first medical image 102 can include a digital radiology image taken to reveal the internal structures of subjects, or to detect certain cells of interest (e.g., cancer cells), without performing invasive procedures. In the example of FIG. 1A, first medical image 102 can be a PET image obtained from a PET scan of the subject's body 106 after the subject receives an injection of a radiolabeled glucose tracer. First medical image 102 may include an activated region 108 having an elevated radioactive level from the radiolabeled glucose tracer, which can indicate the presence of a tumor in the subject's body 106. In addition, second medical image 104 can include a digital pathology image of a specimen 110 prepared from a tissue removed from body 106 of the subject. The specimen can be stained to provide histology information of cells in the specimen. For example, in the example of FIG. 1A, specimen 110 may include a region of tumor cells 112 that can be revealed through staining and captured in second medical image 104.

Due to their different modalities, first medical image 102 and second medical image 104 are typically sourced by a medical information system 120 (e.g., a digital imaging and communications in medicine (DICOM) system) from different databases, and are displayed as two discrete pieces of medical data in different interfaces to be analyzed by different specialists. For example, first medical image 102 can be sourced from a digital radiology image database 130 and displayed in a radiology image interface 132 to a radiologist, whereas second medical image 104 can be sourced from a digital pathology image database 140 and displayed in a pathology image interface 142 to a pathologist. The databases may include, for example, an EMR (electronic medical record) system, a PACS (picture archiving and communication system), a Digital Pathology (DP) system, an LIS (laboratory information system), an RIS (radiology information system), etc.

Currently, medical information system 120 typically does not allow a user to efficiently correlate between two medical images of different modalities, but such a correlation operation may reveal additional information that can facilitate the clinical analysis and/or clinical intervention. For example, medical information system 120 typically does not provide information to assist a user in correlating first medical image 102 and second medical image 104. For example, medical information system 120 typically does not indicate the relationship between activated region 108 and region of cells 112, such as whether they correspond to the same tissue and to the same set of cells. Moreover, medical information system 120 typically does not provide easy and intuitive ways to store and access the correspondence information.

As described above, the correlation between first medical image 102 and second medical image 104 can reveal additional information that can support a clinical diagnosis and/or a clinical intervention. Generating the correlation, or at least providing easy and intuitive ways to store and access the correspondence information, can facilitate the clinical diagnosis and/or the clinical intervention. FIG. 1B illustrates examples of operations that can be supported by the correlation between first medical image 102 and second medical image 104. As shown in FIG. 1B, a cancer diagnosis 150 can be made based on correlating activated region 108 with region of cells 112. For example, activated region 108 may indicate the likely presence of a tumor that metabolizes a radiolabeled glucose tracer. If region of cells 112 and activated region 108 correspond to the same tissue and to the same set of cells, such a correlation can confirm cancer diagnosis 150 for the subject, as well as the location of the cancerous cells/tumor in the subject.

As another example, the correlation can also be used to support a surgical procedure verification 152. Specifically, first medical image 102 may be taken for a subject prior to a surgery to remove a tissue including a tumor, whereas second medical image 104 may be taken for removed tissue after the surgery. If region of cells 112 and activated region 108 correspond to the same tissue and to the same set of cells, it can be determined that the surgery correctly removes the tumor rather than a healthy tissue.

As another example, the correlation can also be used to support a classification operation 154. Specifically, based on region of cells 112 and activated region 108 corresponding to the same tissue and to the same set of cells, as well as knowledge of which part of body 106 is captured in first medical image 102, the source of specimen 110 captured in second medical image 104 can be classified. For example, if the prostate of body 106 is captured in first medical image 102, specimen 110 can be classified as belonging to the prostate. Such information can in turn refine the analysis on second medical image 104. For example, by determining that specimen 110 is a prostate tissue, second medical image 104 can be processed to detect specific patterns associated with tumors associated with prostate cancer, rather than other types of cancers (e.g., lung cancer).

As yet another example, the correlation can also be used to support a research operation 156. The correlation can be made between the medical images of a cohort of subjects to support a research operation, such as a drug discovery research, a translational research to determine the responses of the subjects to a particular treatment, how a particular treatment works in the subjects, etc.

### II. Example Multi-Modal Medical Images Correlating System

### A. System Overview

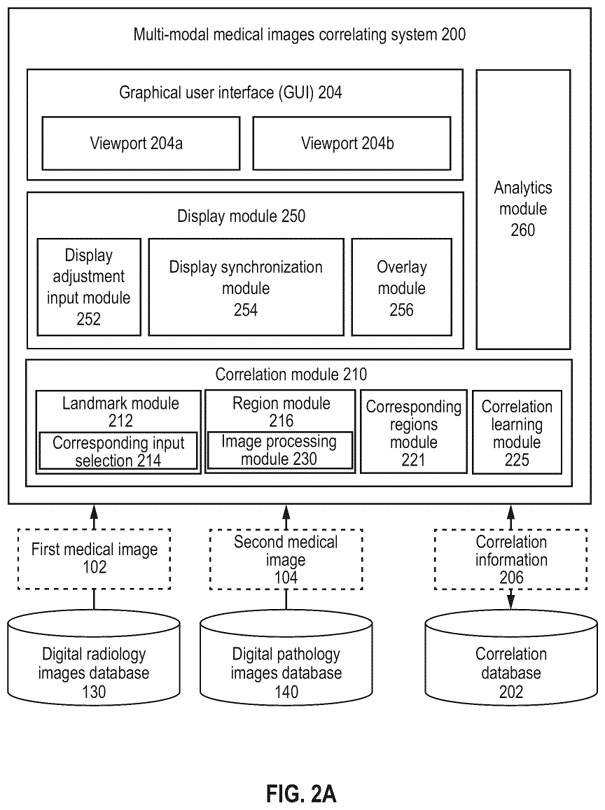

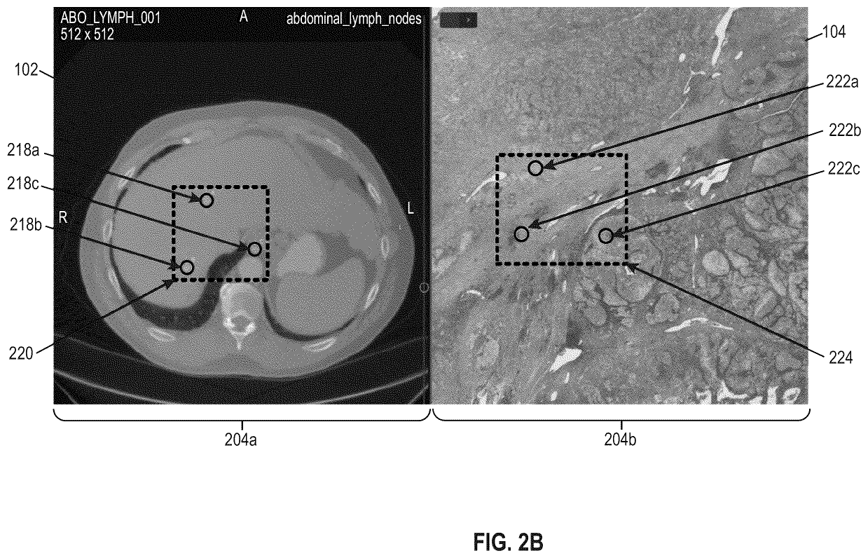

FIG. 2A illustrates an example multi-modal medical images correlating system 200 that can provide access to the correspondence information between regions of interests in multi-modal images. Multi-modal medical images correlating system 200 can be a software system that can access first medical image 102 and second medical image 104 from, respectively, digital radiology images database 130 and digital pathology images database 140. Multi-modal medical images correlating system 200 can determine a correlation between a first region of interest (e.g., activated region 108) in first medical image 102 and a second region of interest (e.g., region of cells 112) in second medical image 104. The correlation can be determined based on a selection input from the user to select corresponding regions of interest between first medical image 102 and second medical image 104, and/or performing correlation analyses on the images to identify corresponding regions of interest. Multi-modal medical images correlating system 200 can generate correspondence information 206 indicating corresponding regions of interest in first medical image 102 and second medical image 104, and store correspondence information 206 at a correlation database 202 to provide easy access to the correspondence information when first medical image 102 and second medical image 104 are accessed again in the future. In addition, multi-modal medical images correlating system 200 further includes a graphical user interface (GUI) 204 that can accept the inputs from the user for correlation determination. GUI 204 can also include multiple viewports, such as viewports 204a and 204b, to display first medical image 102 and second medical image 104 simultaneously. GUI 204 can also detect a display adjustment input from the user in one of the viewports (e.g., viewport 204a), and synchronize an adjustment of the display of both first medical image 102 and second medical image 104 in their respective viewports to facilitate visual comparison/correlation between the corresponding regions of interest between first medical image 102 and second medical image 104.

### B. Correlation Based on Landmarks

Specifically, multi-modal medical images correlating system 200 includes a correlation module 210. Correlation module 210 can determine a correlation between a first region of interest (e.g., activated region 108) in first medical image 102 and a second region of interest (e.g., region of cells 112) in second medical image 104. The correlation can be determined based on a selection input from the user to select corresponding regions of interest between first medical image 102 and second medical image 104, and/or performing correlation analyses on the images to identify corresponding regions.

In some examples, correlation module 210 includes a landmark module 212 to receive the selection inputs as landmarks. The landmarks can be points in a medical image to indicate a certain feature of interest selected by the user and to be encompassed by a region of interest. The selection inputs can be received via viewports 204a and 204b on the displayed medical images. Landmark inputting module 212 can then determine the image locations (e.g., pixel coordinates) of the selected landmarks in the medical images. In some examples, landmark inputting module 212 can provide a corresponding input selection option 214 via GUI 204 to receive selection of landmarks in both first medical image 102 (via viewport 204a) and second medical image 104 (via viewport 204b). The selected landmarks in first medical image 102 and second medical image 104 via the selection option can indicate the regions of interest that encompass the selected landmarks in the two medical images corresponding to each other (e.g., corresponding to the same tissue and to the same set of cells).

In addition, correlation module 210 includes a region module 216 to determine a region of interest in each of first medical image 102 and second medical image 104. In a case where the regions of interest are determined based on landmarks input via GUI 204, region module 216 can determine the regions of interest to encompass the landmarks. The region of interest can be of any pre-determined shapes (e.g., triangle, rectangle, oval, etc.), and the boundaries of the region of interest can be of pre-determined distance from the landmarks. In some examples, corresponding regions module 216 can also adjust the shape of the region of interest based on the number of landmarks selected. For example, if the number of landmarks exceed a pre-determined threshold number, corresponding regions module 216 can determine a polygon region of interest, with the landmarks becoming the vertices of the polygon region.

Correlation module 210 further includes a corresponding regions module 221 to determine that two regions of interest in first medical image 102 and second medical image 104 are corresponding regions of interest (e.g., corresponding to the same tissue and/or same region of cells). In some examples, corresponding regions module 221 can determine that two regions of interest correspond to each other based on a user's input. For example, if the landmarks are selected via corresponding input selection option 214 in first medical image 102 and second medical image 104, corresponding regions module 216 can designate the selected landmarks in each medical image to correspond to each other, and that the regions of interest encompassing the selected landmarks also correspond to each other between the two medical images.

FIG. 2B illustrates examples of landmarks and regions of interest displayed by GUI 204. As shown in FIG. 2B, viewport 204a can display first medical image 102, landmarks 218a, 218b, and 218c selected by a user via GUI 204, as well as a first region of interest 220 that encompasses landmarks 218a, 218b, and 218c. The landmarks can be shown as annotations in the GUI. In addition, viewport 204b can display second medical image 104, landmarks 222a, 222b, and 222c selected by the user via GUI 204, as well as a second region of interest 224 that encompass landmarks 222a, 222b, and 222c. The landmarks and the boundaries of the regions of interest are displayed as indications of the regions of interest. If the landmarks are selected via corresponding input selection option 214, corresponding regions module 216 can designate that landmark 218a as corresponding to landmark 222a, landmark 218b as corresponding to landmark 222b, landmark 218c as corresponding to landmark 222c, and first region of interest 220 as corresponding to second region of interest 224.

Landmark module 212 can receive selection of the landmarks via GUI 204 and viewports 204a and 204b, and determine the pixel locations of the landmarks based on, for example, locations of the selection in the viewport with respect to the scale of the medical image displayed. For example, viewport 204a can determine the display scaling factor of first medical image 102 in the viewport. The selection locations, as well as the locations of landmarks 218a-218c, in viewport 204a can then be translated to pixel locations within first medical image 102 based on the display scaling factor.

### C. Correspondence Information

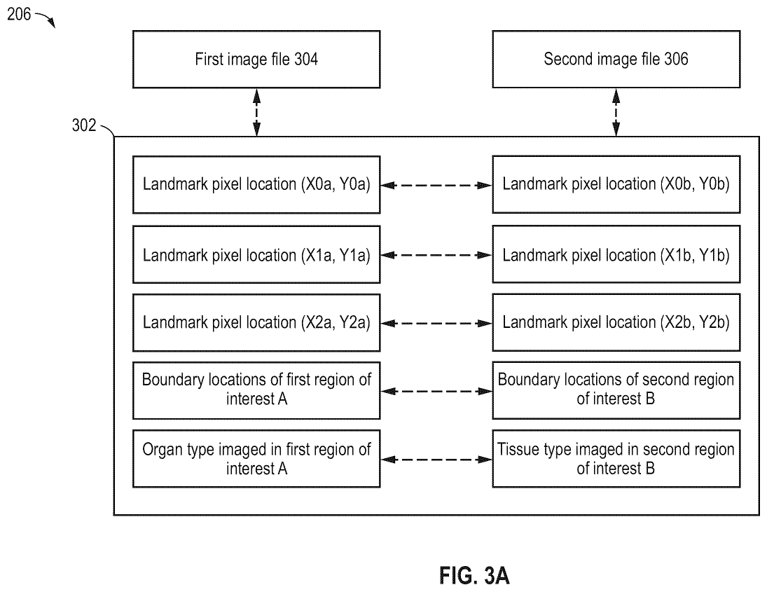

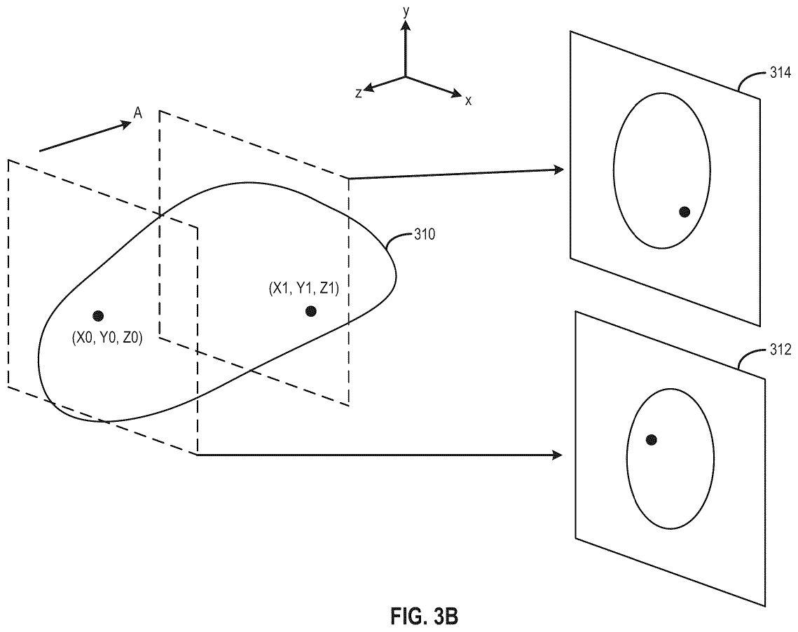

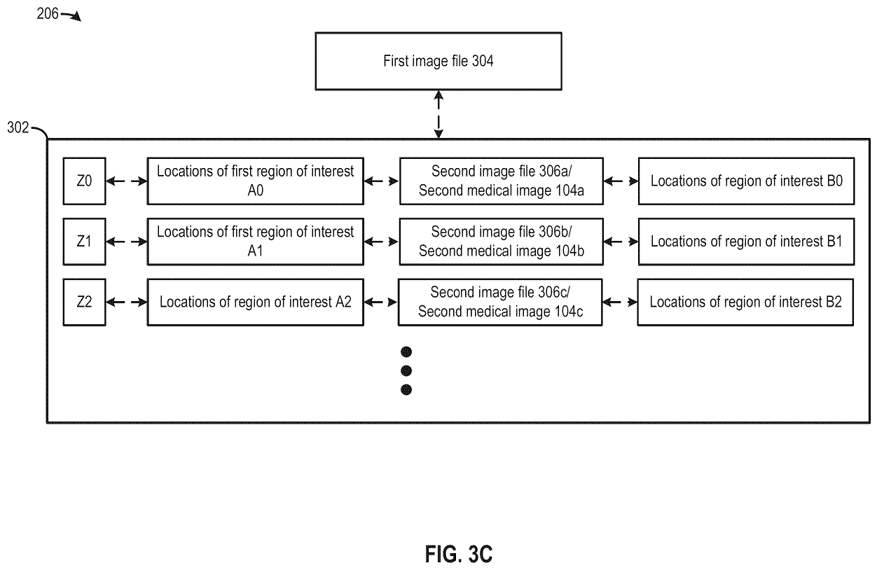

After determining corresponding landmarks and regions of interest between first medical image 102 and second medical image 104, correlation module 210 can generate correspondence information 206 to store data indicating the correspondence between the landmarks and regions of interest. FIG. 3A illustrates an example of correspondence information 206. As shown in FIG. 3A, correspondence information 206 can include a data structure 302 that maps between first locations of a first region of interest (e.g., first region of interest A in FIG. 3A) and second locations of a second region of interest (e.g., second region of interest B in FIG. 3A). Data structure 302 may include, for example, a mapping table. In some examples, the first locations and second locations can include the actual pixel locations of the selected landmarks, such as (X0a, Y0a), (X1a, Y1a), and (X2a, Y2a) in first medical image 102, (X0b, Y0b), (X1b, Y1b), and (X2b, Y2b) in second medical image 104, as well as boundary locations of the regions of interest in first medical image 102 and second medical image 104. Data structure 302 may include additional information, such as the type of organ (e.g., lung, kidney, prostate, etc.) captured in the region of interest A and the type of tissue (e.g., lung cell, prostate cell, tumor cell, etc.) captured in the region of interest B. Correspondence information 206 can also include a reference (e.g., file name, pointer, etc.) to first and second image files 304 and 306 including, respectively, first medical image 102 and second medical image 104. Such arrangements can link the regions of interest information, including the regions' locations and the correspondence relationship, to the electronic files, which allows multi-modal medical images correlating system 200 to access correspondence information 206 upon accessing the electronic files of first medical image 102 and second medical image 104.

In some examples, first medical image 102 can be part of a 3D PET image that comprises multiple 2D PET images obtained at different longitudinal positions. In addition, multiple second medical images can also be generated from slicing a tissue at different longitudinal positions along the Z-axis. FIG. 3B illustrates an example of generating multiple medical images from a tissue mass 310. As shown in FIG. 3B, a 3D PET scanning operation can be performed along a scanning direction A. Multiple 2D PET images, which can include images 312 and 314, can be obtained at different longitudinal locations Z0 and Z1 along the Z-axis. Tissue mass 310 can include a first region at a first location (X0, Y0, and Z0) captured in image 312, and a second region at a second location (X1, Y1, and Z1) captured in image 314. In addition, images 312 and 314 can also be generated as digital pathology images from tissue specimens obtained from slicing tissue mass 310 at locations Z0 and Z1 along the Z-axis.

In a case where first medical image 102 includes multiple 2D PET images, data structure 302 can map multiple first regions of interest in the multiple 2D PET images to multiple second regions of interest in multiple digital pathology images of second medical images 104. FIG. 3C illustrates an example of data structure 302 that maps first medical image 102 to multiple second medical images 104. Referring to FIG. 3C, first medical image 102, associated with first image file 304, may include a first 2D PET image captured at longitudinal position Z0, a second 2D PET image captured at longitudinal position Z1, a third 2D PET image captured at longitudinal position Z2, etc. The first 2D PET image can include a first region of interest A0, the second 2D PET image can include a first region of interest A1, and the third 2D PET image can include a first region of interest A2. In addition, there can be multiple second medical images including second medical images 104a, 104b, and 104c, each including, respectively, second regions of interest B0, B1, and B2. Data structure 302 can provide a mapping among a longitudinal position (e.g., one of Z0, Z1, or Z2), locations of a first region of interest in the 2D PET image captured at that longitudinal position (e.g., one of A0, A1, or A2), a second medical image (e.g., one of 104a, 104b, or 104c) from the tissue slide obtained at that longitudinal position and its associated file (e.g., one of 306a, 306b, or 306c), as well as locations of a second region of interest in the second medical image (one of B0, B1, or B2). With such arrangements, when a user navigates through different 2D PET scan images via multi-modal medical images correlating system 200 representing different longitudinal positions, the system can retrieve the corresponding second image files 306 and display the second medical images 104 included in the files. Moreover, multi-modal medical images correlating system 200 can also display the indications of the regions of interest in each medical images (e.g., landmarks, boundary lines, etc.) based on the locations of regions of interest information in data structure 302. In addition, as to be described below, the 3D locations information in data structure 302 can also support various display effects, such as 3D rotations.

### D. Automatic Correlation

In some examples, in additional to (or in lieu of) receiving a user input, correlation module 210 can also determine corresponding regions of interest automatically from first medical image 102 and second medical image 104. Specifically, referring back to FIG. 2A, region module 216 can include an image processing module 230 to perform image processing operations on first medical image 102 and second medical image 104, and determine a region of interest in each image based on the image processing operations results.

For example, in a case where first medical image 102 is a PET image, image processing module 230 can compare the radioactive level revealed at each pixel of the PET image with a threshold, and region module 216 can include pixels having the radioactive level exceeding the threshold in the first region of interest in first medical image 102. In addition, in a case where second medical image 104 is a digital pathology image taken from a stained specimen slide, image processing module 230 can perform a feature extraction operation to detect features that represent cells of interest, such as specific stain patterns indicative of a particular type of cancer cells and/or cell structures, specific fluorescent tagging that reveals a particular layer of cell/cell structure, etc. Region module 216 can include pixels that reveal such straining patterns in the second region of interest in second medical image 104.

Upon determining the first and second regions of interest, region module 216 can provide the first and second regions of interest for display in viewports 204a and 204b. In some examples, the first and second regions of interest can be output as candidate regions of interest, and GUI 204 can prompt the user to confirm whether the first and second regions of interest correspond to each other. Upon receiving confirmation from the user, correlation module 210 can store the information of the first and second regions of interest as part of correspondence information 206 shown in FIG. 3A and FIG. 3C.

### E. Convolutional Neural Network

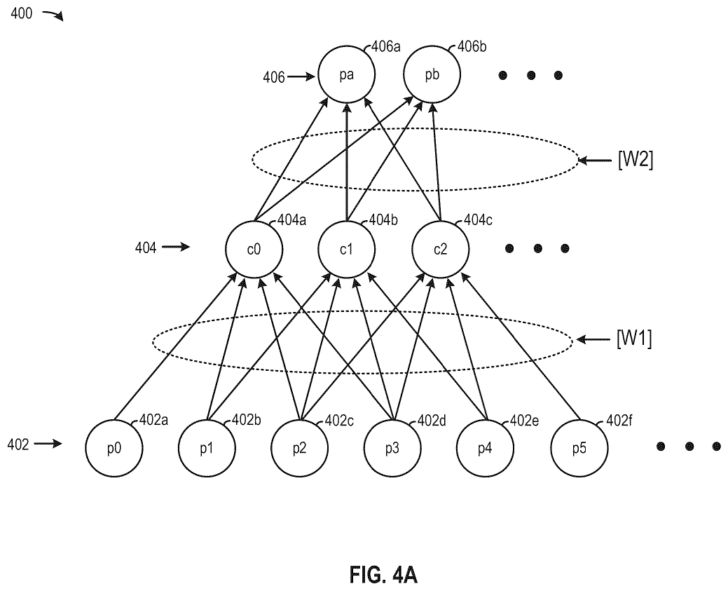

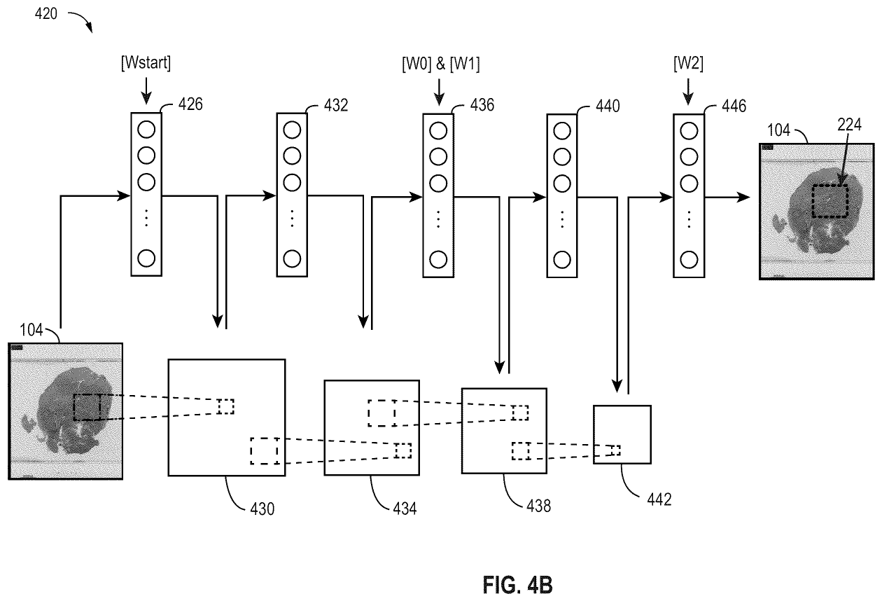

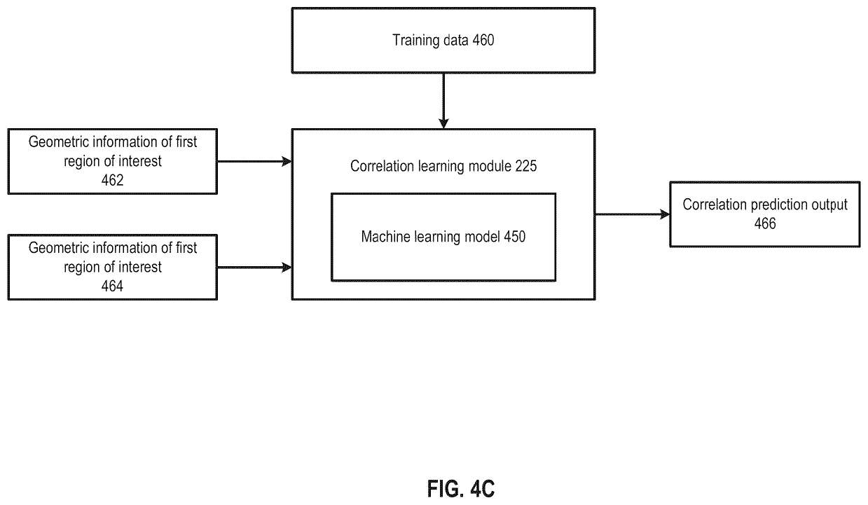

In some examples, image processing module 230 can implement a machine learning model, such as a convolutional neural network, to perform feature extraction operations. FIG. 4A and FIG. 4B illustrate examples of a convolutional neural network (CNN) 400 that can be part of image processing module 230. FIG. 4A illustrates a simplified version of CNN 400. As shown in FIG. 4A, CNN 400 includes at least an input layer 402, a middle layer 404, and an output layer 406. Input layer 402 and middle layer 404 together can perform a convolution operation, whereas output layer 406 can compute probabilities of a tile (e.g., a two-dimensional array with N×M dimensions) of pixels being classified into each of candidate prediction outputs.

Specifically, input layer 402 can include a set of input nodes, such as input nodes 402a, 402b, 402c, 402d, 402e, and 402f. Each input node of input layer 402 can be assigned to receive a pixel value (e.g., p0, p1, p2, p3, p4, p5, etc.) from a medical image, such as medical image 102, and scale the pixel based on a weight of a weight array [W1]. Weight array [W1] can be part of a kernel and can define the image features to be detected in the pixels.

In addition, middle layer 404 can include a set of middle nodes, including middle nodes 404a, 404b, and 404c. Each middle node can represent a tile of pixels and can receive the scaled pixel values from a group of input nodes that overlap with the kernel. Each middle node can sum the scaled pixel values to generate a convolution output. For example, middle node 404a can generate a convolution output c0 based on scaled pixel values p0, p1, p2, and p3; middle node 404b can generate a convolution output c1 based on scaled pixel values p1, p2, p3, and p4; and middle node 404c can generate a convolution output c2 based on scaled pixel values p2, p3, p4, and p5. Each middle node can scale the convolution output with a set of weights defined in a weight array [W2]. Weight array [W2] can define a contribution of a convolution output to the probability of a tile being classified into one of the candidate prediction outputs. Weight array [W2] can also be part of a kernel.

Output layer 406 includes one or more nodes, including 406a, 406b, etc. Each node can correspond to a tile and can compute the probability of the tile being classified into a prediction output. For example, in a case where CNN 400 is used to predict whether a tile of pixels is part of a tumor, each of nodes 406a, 406b, etc., can output a probability (e.g., pa, pb, etc.) of the corresponding tile being classified into a tumor. Region module 216 can then include tiles of pixels having the probabilities exceeding a threshold into the second region of interest of second medical image 104.

FIG. 4B illustrates additional details of a CNN 420. As shown in FIG. 4B, CNN 420 may include four main operations: (1) convolution; (2) non-linear activation function (e.g., ReLU); (3) pooling or sub-sampling; and (4) classification.

As shown in FIG. 4B, second medical image 104 may be processed by a first convolution network 426 using a first set of weight arrays (e.g., [Wstart] in FIG. 4B). As part of the convolution operation, blocks of pixels of medical image 102 can be multiplied with the first weights array to generate a sum. Each sum is then processed by a non-linear activation function (e.g., ReLU, softmax, etc.) to generate a convolution output, and the convolution outputs can form an output matrix 430. The first weights array can be used to, for example, extract certain basic features (e.g., edges, etc.) from medical image 102, and output matrix 430 can represent a distribution of the basic features as a basic feature map. Output matrix (or feature map) 430 may be passed to a pooling layer 432, where output matrix 430 may be subsampled or down-sampled by pooling layer 432 to generate a matrix 434.

Matrix 434 may be processed by a second convolution network 436, which can include input layer 402 and middle layer 404 of FIG. 4A, using a second weights array (e.g., [W1] and [W2] in FIG. 4A). The second weights array can be used to, for example, identify stain patterns for a cancer cell. As part of the convolution operation, blocks of pixels of matrix 434 can be multiplied with the second weights array to generate a sum. Each sum is then processed by a non-linear activation function (e.g., ReLU, softmax, etc.) to generate a convolution output, and the convolution outputs can form an output matrix 438. A non-linear activation function (e.g., ReLU) may also be performed by the second convolution network 436 as in the first convolution layer. An output matrix 418 (or feature map) from second convolution network 436 may represent a distribution of features representing a type of organ. Output matrix 438 may be passed to a pooling layer 440, where output matrix 418 may be subsampled or down-sampled to generate a matrix 442.

Matrix 442 can then be passed through a fully-connected layer 446, which can include a multi-layer perceptron (MLP). Fully-connected layer 446 can perform a classification operation based on matrix 442. The classification output can include, for example, probabilities of a tile being classified into a cancer cell, as described in FIG. 4A. Fully connected layer 446 can also multiply matrix 442 with a third weight array (labelled [W2]) to generate sums, and the sums can also be processed by an activation function (e.g., ReLu, softmax, etc.) to generate a distribution of probabilities. Based on the distribution of probabilities, region module 216 can determine second region of interest 224.

### F. Learning-Based Correlation

In addition to feature extraction and region of interest determination, correlation module 210 can also automate the determination of whether two regions of interest correspond to each other. Referring back to FIG. 2A, correlation module 210 can include a correlation learning module 225 that can learn from other correlated pairs of regions of interest to perform the correlation determination. FIG. 4C illustrates an example operation of correlation learning module 225. As shown in FIG. 4C, correlation module 210 can include a machine learning model 450 (e.g., a neural network, a decision tree, etc.) that can be trained by training data 460 including pairs of corresponding regions of interest. Training data 460 can include, for example, geometric information such as shapes, sizes, and pixel locations of corresponding regions of interest. The trained machine learning model 450 can then be employed by correlation module 210. Machine learning model 450 can receive, as inputs, geometric information 462 of a first region of interest and geometric information 464 of a second region of interest, and generate a correlation prediction output 466 of whether the first region of interest and the second region of interest correspond to each other.

### G. Display Module

Referring back to FIG. 2A, multi-modal medical images correlating system 200 further includes a display module 250 to control the display of first medical image 102 in viewport 204a and the display of second medical image 104 in viewport 204b. Specifically, multi-modal medical images correlating system 200 includes a display adjustment input module 252, a display synchronization module 254, and an overlay module 256.

Display adjustment input module 252 can receive a display adjustment input while viewport 204a displays first medical image 102 and viewport 204b displays second medical image 104. The display adjustment input can be received via one of viewports 204a or 204b to adjust the displaying of one of first region of interest 220 or second region of interest 224 in the viewport that receives the input. The display adjustment input can include, for example, a zoom-in/zoom-out input, a panning input, a rotation input, etc. Display synchronization module 254 can determine the adjustment of the displaying of a region of interest at the viewport that receives the input, and adjust the displaying of the region of interest at that viewport. In addition, as part of adjustment synchronization, display synchronization module 254 can also adjust the displaying of the other region of interest at the other viewport that does not receive the input, and the adjustment is made based on the input as well as the geometric information (e.g., pixel location, size, shape, etc.) of the other region of interest. As a result of the synchronization, various settings of the displaying, such as a degree of magnification, the portion of the region of interest selected for display, a viewpoint of the region of interest, etc., are applied to both viewports.

Such arrangements can facilitate visual comparison/correlation between the two regions of interest, which in turn can facilitate a clinical diagnosis based on the medical images. For example, first region of interest 220 may represent part of a body having an elevated radioactive level (from the radiolabeled glucose tracer), which can indicate the presence of a tumor, while second region of interest 224 may reveal stain patterns of tumor cells or of healthy cells. A visual comparison between the two regions of interest can confirm the presence of a tumor, and/or verify that a prior cancer surgery has removed a cancerous tissue other than a healthy tissue.

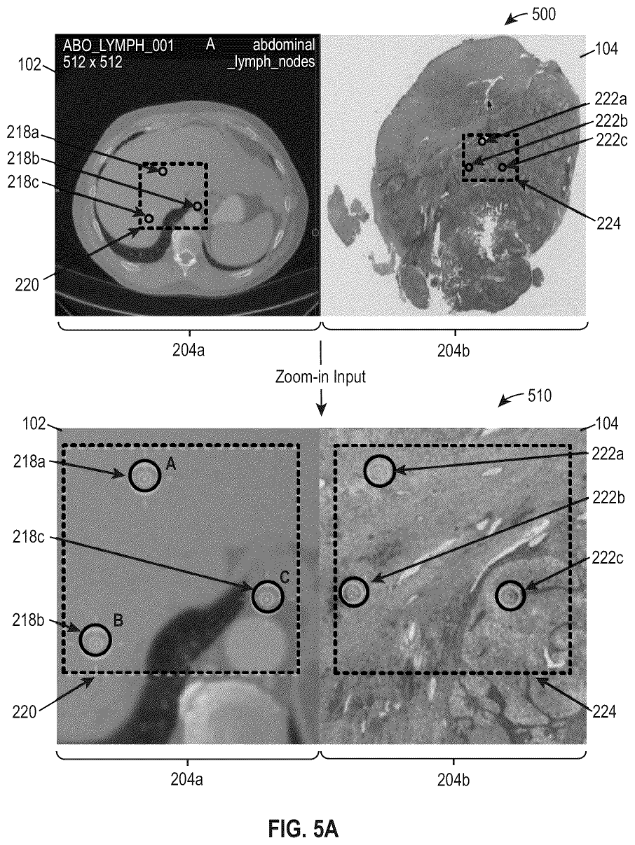

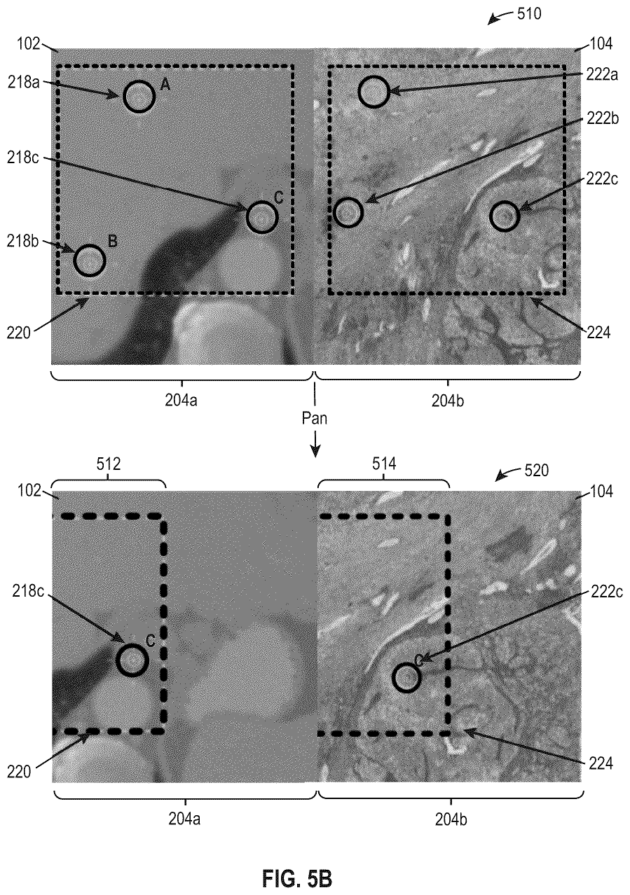

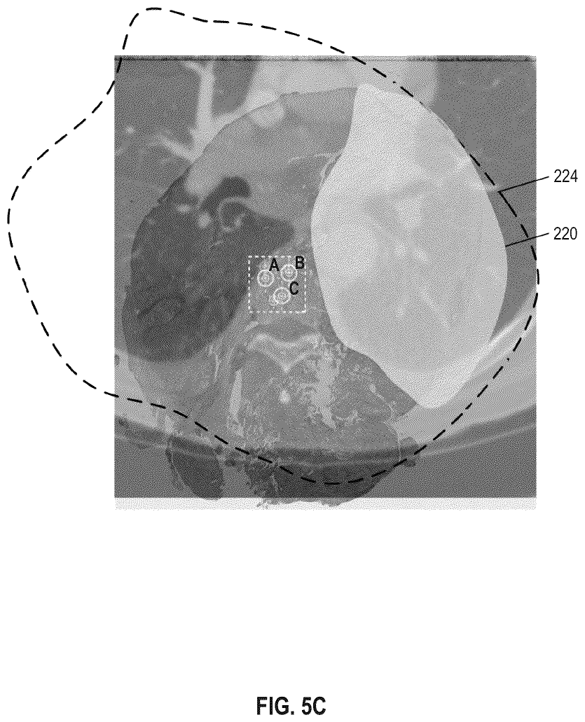

FIG. 5A illustrates an example of a synchronized zoom operation. In state 500, viewport 204a can display first medical image 102, as well as landmarks 218a-c and first region of interest 220 with a first display scaling factor. Moreover, viewport 204b can also display second medical image 104, as well as landmarks 222a-c and second region of interest 224 with second display scaling factors. First medical image 102 and second medical image 104 can be displayed with different display scaling factors and at different degrees of magnification. Therefore, first region of interest 220 and second region of interest 224 can be displayed as having different sizes.

Viewport 204a can receive a zoom-in input to zoom into the first region of interest 220 in viewport 204a, and both viewports 204a and 204b can transition to state 510. In state 510, based on the zoom-in input, display synchronization module 254 can compute a degree of magnification, and magnify first region of interest 220, as well as some portions of first medical image 102 around first region of interest 220, in viewport 204a by the degree of magnification. In addition, display synchronization module 254 can identify second region of interest 224 and its location in second medical image 104 (e.g., based on correspondence information 206), and magnify second region of interest 224 by the same degree of magnification in viewport 204b. Due to the same degree of magnification, first region of interest 220 and second region of interest 224 are displayed to the same scale and have the same size.

FIG. 5B illustrates an example of a synchronized panning operation. Based on a pan input (e.g., a pan-left/pan-right/pan-up/pan-down input), viewports 204a and 204b can transition from state 510 of FIG. 5A to state 520, in which viewport 204a displays a right portion 512 of first region of interest 220 as well as landmark 218c. In addition, as part of state 520, viewport 204b displays a right portion 522 of second region of interest 224 as well as landmark 222c. Right portion 512 of first region of interest 220 can be displayed as of the same scale and degree of magnification as right portion 522 of second region of interest 224, such that the same extent/portion of a region of interest, as well as corresponding landmarks 218c and 222c, are displayed in both viewports.

In addition to zoom and panning operations, display synchronization module 254 can also synchronize the rotation (2D or 3D) of the regions of interest in both viewports. For example, display synchronization module 254 may receive an input to rotate first region of interest 220 (and first medical image 102) by a certain degree in viewport 204a. Based on the input, display synchronization module 254 can cause viewport 204b to rotate second region of interest 224 (and second medical image 104) by the same degree.

In addition to synchronizing the displaying of first region of interest 220 and second region of interest 224 in their respective viewports, display module 250 can also perform other types of display operations. For example, display module 250 includes an overlay module 256 to control a viewport (e.g., viewports 204a, 204b, or another viewport) to display both first medical image 102 and second medical image 104 in that viewport, with first region of interest 220 and second region of interest 224 displayed to the same scale and one region of interest overlaying over the other. FIG. 5C illustrates an example in which first region of interest 220 is overlaid on second region of interest 224, and first region of interest 220 is made in a semi-transparent form. Such arrangements can further support visual comparison between first region of interest 220 and second region of interest 224, which in turn can facilitate a clinical diagnosis based on the medical images as described above.

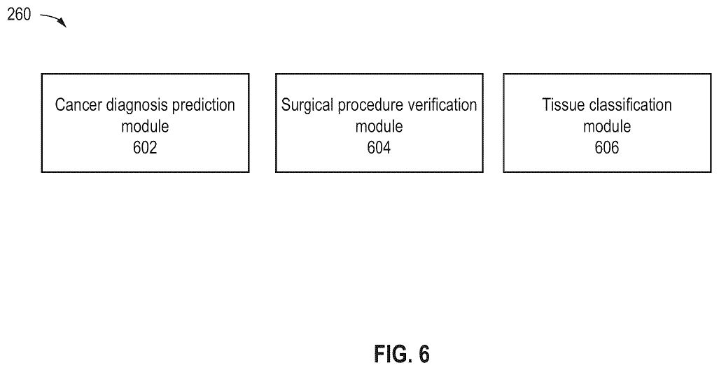

Referring back to FIG. 2A, in addition to displaying the medical images, multi-modal medical images correlating system 200 may further include an analytics module 260 to perform additional analyses based on the outputs of correlation module 210. FIG. 6 illustrates examples of internal components of analytics module 260. As shown in FIG. 6, analytics module 260 may include a cancer diagnosis module 602, a surgical procedure verification module 604, and a tissue classification module 606.

Cancer diagnosis module 602 can output a diagnosis prediction based on a correlation between first region of interest 220 and second region of interest 224. For example, first region of interest 220, which can be in a PET scan image, may be identified based on having an elevated radioactive level (from the radiolabeled glucose tracer), which can indicate the presence of a tumor. Second region of interest 224 may include a stain pattern that is determined to include cancer cells. If corresponding regions module 221 determines that these regions of interest correspond to each other, cancer diagnosis module 602 can output a cancer diagnosis prediction for the subject, as well as the location of the cancerous cells/tumor in the subject.

In addition, surgical procedure verification module 604 can perform a surgical procedure verification operation based on a correlation between first region of interest 220 and second region of interest 224. Specifically, first medical image 102 may be taken for a subject prior to a surgery to remove a tissue including a tumor, and the tumor is detected and captured in first region of interest 220. In addition, second medical image 104 may be taken for removed tissue after the surgery, and suspected cancer cells are detected and captured in second region of interest 222. If corresponding regions module 221 determines that first region of interest 220 and second region of interest 224 correspond to each other, surgical procedure verification module 604 can generate a verification output indicating that the surgery is likely to correctly remove the tumor rather than a healthy tissue.

Further, tissue classification module 606 can perform a tissue classification operation based on a correlation between first region of interest 220 and second region of interest 224. For example, first medical image 102, which includes first region of interest 220, may include metadata that indicates the type of tissue/organ, or which part of the subject's body, is captured in the medical image. If corresponding regions module 221 determines that first region of interest 220 and second region of interest 224 correspond to each other, tissue classification module 606 can classify the tissue captured in second medical image 104 based on the metadata of first medical image 102. In a case where second medical image 104 also includes metadata that specifies the tissue captured in the image, tissue classification module 606 can also verify the metadata between the two medical images to detect potential inconsistency. The classification can also determine the additional processing of second medical image 104. For example, as described above, if it is determined that the tissue captured in second medical image 104 is a prostate tissue, second medical image 104 can be processed to detect specific patterns associated with tumors associated with prostate cancer, rather than other types of cancers (e.g., lung cancer).

### III. Examples of Graphical User Interface

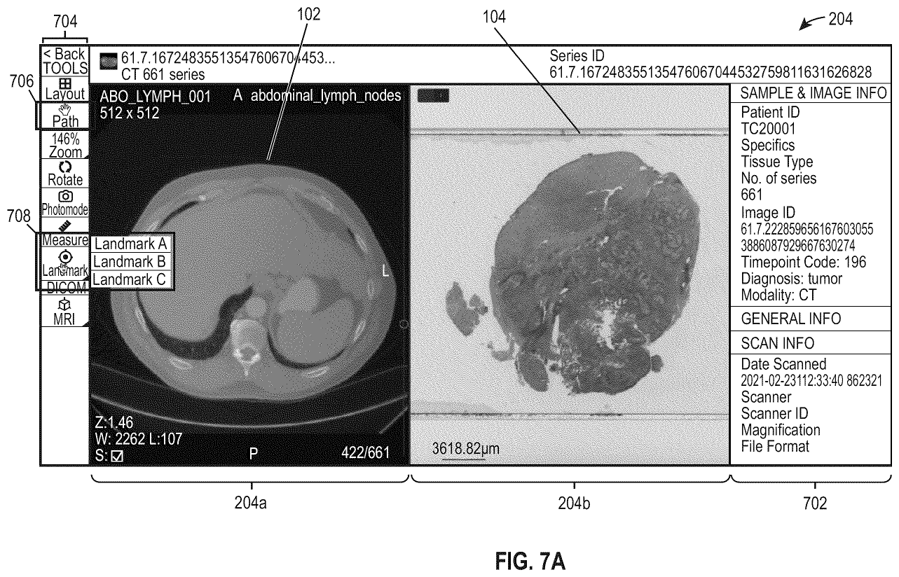

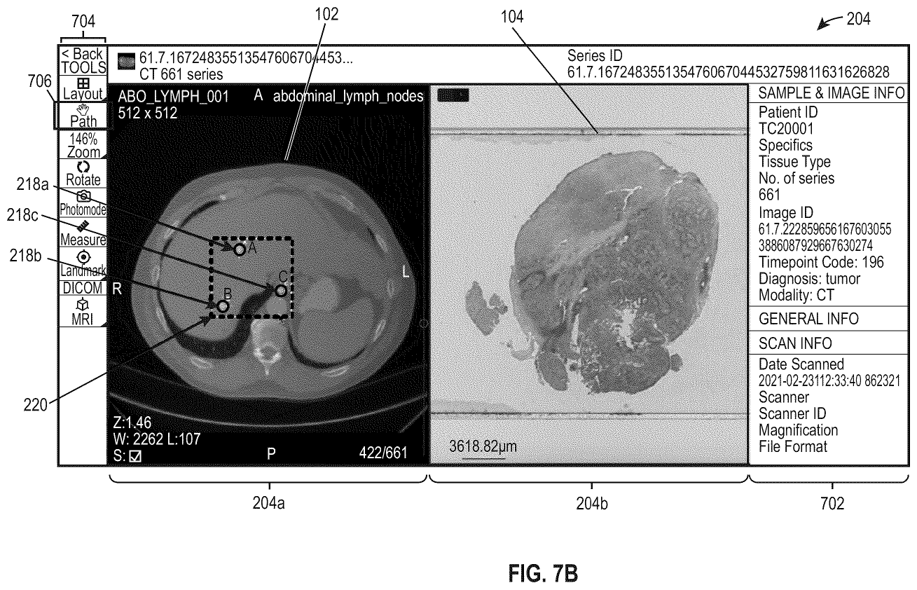

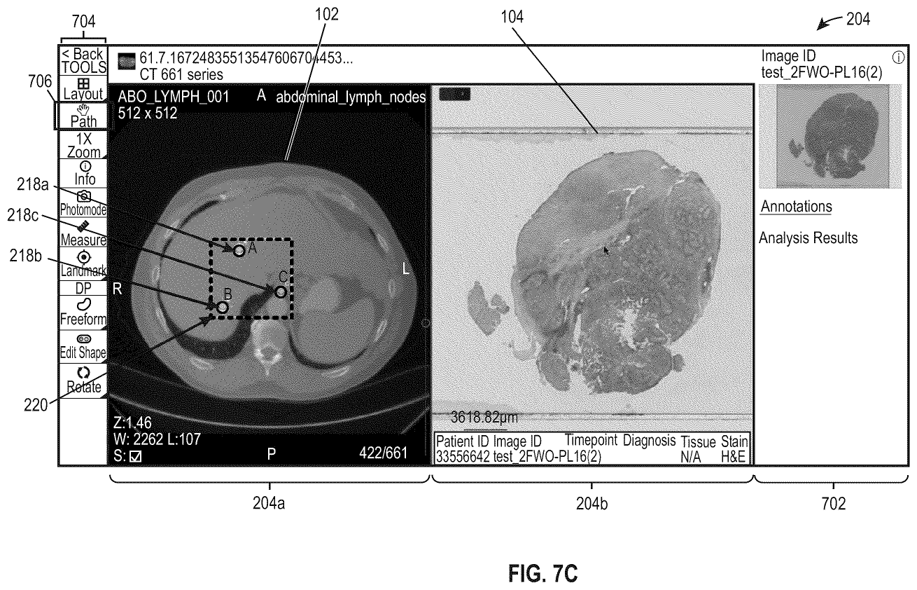

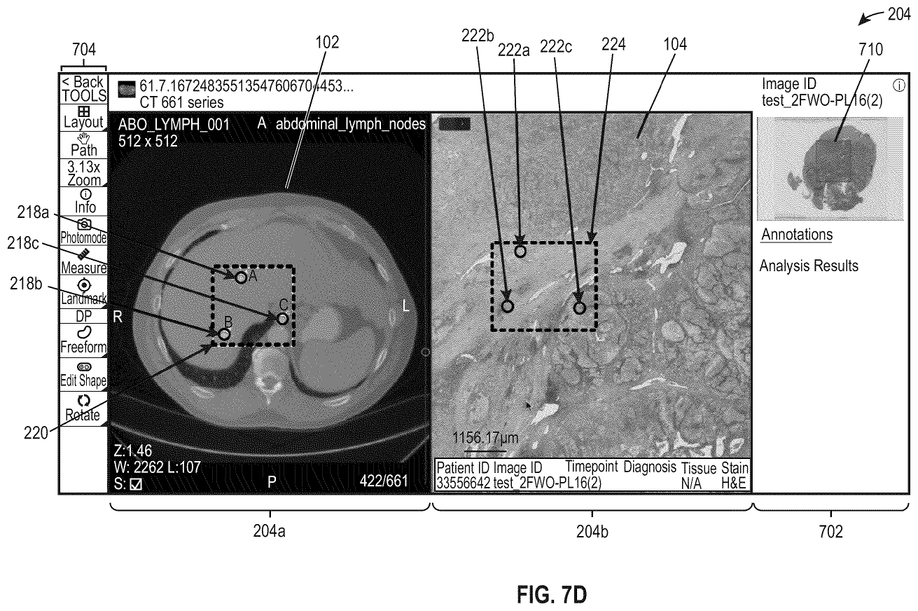

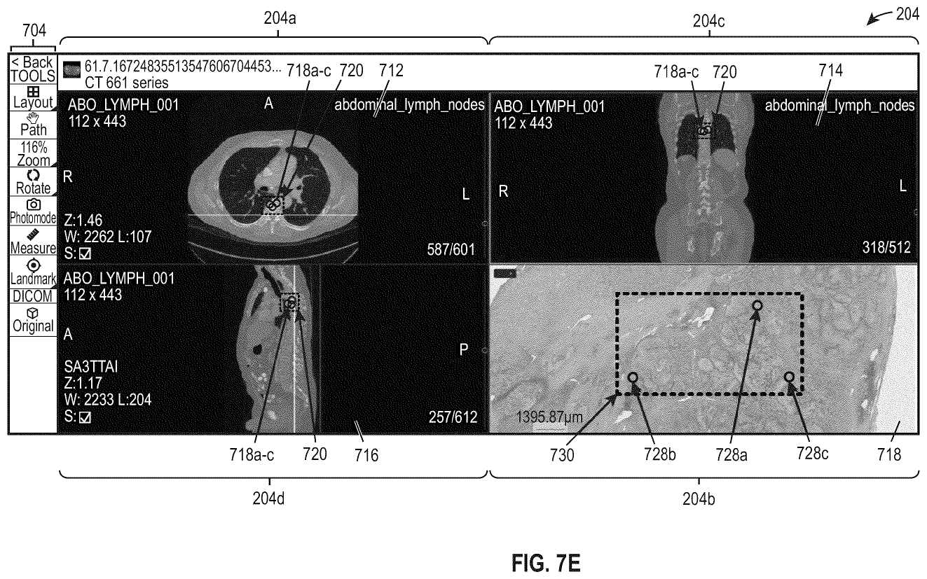

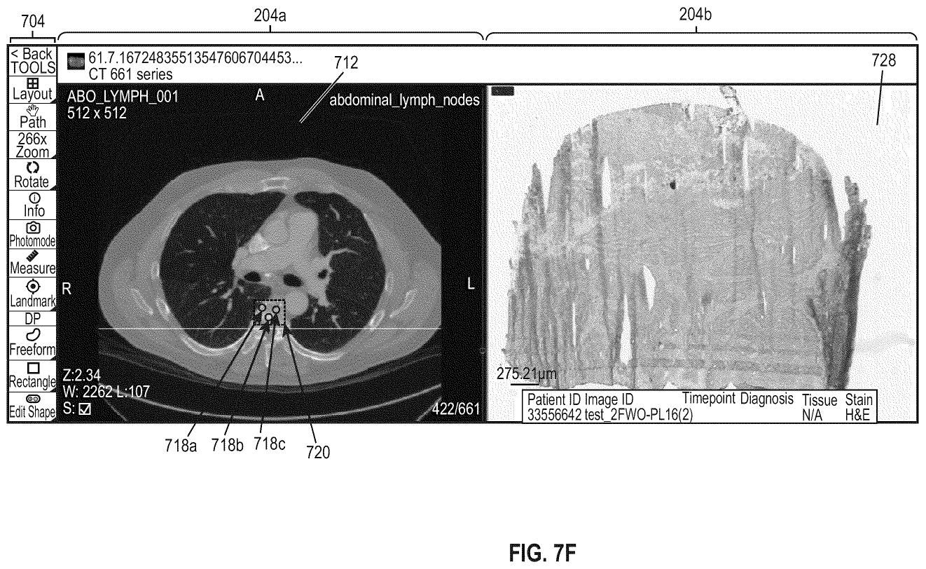

FIG. 7A, FIG. 7B, FIG. 7C, FIG. 7D, FIG. 7E, and FIG. 7F illustrate examples of GUI 204 and its operations supported by multi-modal medical images correlating system 200. As shown in FIG. 7A, GUI 204 includes viewport 204a, viewport 204b, a side panel 702, and an options menu 704. In the example shown in FIG. 7A, viewport 204a displays first medical image 102, whereas viewport 204b displays second medical image 104. The medical images can be displayed in different scales. Side panel 702 can display some of the information included in the metadata of both images, such as information of the subject, the source of the images, etc. Options menu 704 includes a zoom-in option 706 to zoom into one of the images at one of the viewports, and an option 708 to add corresponding landmarks in both images. Option 708 corresponds to corresponding input selection option 214 provided by landmark module 212 of FIG. 2A.

FIG. 7B illustrates a state of GUI 204 after receiving selection of landmarks 218a-218c from the user via option 708. The landmarks can be selected using any input devices, including touch screen, in viewport 204a. Landmark module 212 can determine the pixel locations of the landmarks in first medical image 102 based on the locations where viewport 204a receives the selection, as well as the display scaling factor applied by viewport 204a in displaying first medical image 102. After receiving the selection, region module 216 can determine first region of interest 220 based on the pixel locations of landmarks 218a-218c.

Following the selection of landmarks 218a-218c of first medical image 102 in viewport 204a, GUI 204 can receive the selection of landmarks for second medical image 104 in viewport 204b. The selection of landmarks for second medical image 104 can be performed when second medical image 104 is magnified. For example, referring to FIG. 7C, GUI 204 may detect that zoom-in option 706 is activated to zoom into second medical image 104. GUI 204 can then receive the selection of landmarks for second medical image 104 when the image is displayed in a magnified form. Upon receiving zoom-in option 706, GUI 204 can display second medical image 104 in viewport 204b. GUI 204 can also detect the selection of a region in second medical image 104 to be zoomed into.

In FIG. 7D, GUI 204 can detect the selection of a region 710 in second medical image 104 to be zoomed into. Viewport 204b can then display region 710 of second medical image 104 in a magnified scale (e.g., with a reduced display scaling factor). Viewport 204b can also receive selection of landmarks 222a-222c. Landmark module 212 can determine the pixel locations of the landmarks in second medical image 104 based on the locations where viewport 204b receives the selection, as well as the display scaling factor applied by viewport 204b in displaying second medical image 104 in the magnified scale. After receiving the selection, region module 216 can determine second region of interest 224 based on the pixel locations of landmarks 222a-222c.

FIG. 7E and FIG. 7F illustrate additional examples of GUI 204 in supporting correlation of regions in a 3D PET image and a digital pathology image. Referring to FIG. 7E, GUI 204 can provide, in additional to viewports 204a and 204b, viewports 204c and 204d. Viewports 204a, 204c, and 204d can show different views of a 3D PET image. For example, viewport 204a can show a medical image 712 of an axial/transversal view of a subject's body, viewport 204c can show a medical image 714 of a coronal/frontal view of the subject's body, whereas viewport 204d can show a medical image 716 of a sagittal/longitudinal view of the subject's body. Meanwhile, viewport 204b can show a digital pathology image 718.

GUI 204 may receive selection of landmarks 718a-c to denote a region of interest 720 in the 3D PET image in one of viewports 204a, 204c, or 204d. Upon receiving the selection of the landmarks, GUI 204 can determine the three-dimension coordinates of landmarks 718a-c and region of interest 720. The three-dimension coordinates can be determined based on the two-dimensional pixel coordinates in the medical image in which the landmarks are selected, as well as the location represented by the medical image within the 3D PET image as described above in FIG. 3B. For example, in a case where the landmarks are selected from medical image 712 of an axial view of the subject's body, the longitudinal coordinate can be determined based on the longitudinal location represented by medical image 712 in the 3D PET image. GUI 204 can then translate the three-dimensional coordinates to the pixel coordinates in other medical images shown in other viewports, and show the landmarks and regions of interests in those medical images. GUI 204 can also detect the selection of landmarks 728a-c and a region of interest 730 in digital pathology image 718 shown in viewport 204b, and store the landmarks in the 3D PET image and digital pathology image 718 and their correspondence as part of correlation information 206 in correlation database 202.

In some examples, correlation information 206 may indicate location correspondence between 3D PET images and digital pathology images. The location correspondence can be based on the longitudinal position of each 2D PET images in the 3D PET image, and the longitudinal position of the tissue slide in the subject's body captured in the digital pathology image. Based on the correlation information, GUI 204 can retrieve a corresponding pair of 2D PET image and digital pathology image. For example, as shown in FIG. 7F, based on correlation information 206, GUI 204 can determine that digital pathology image 728 corresponds to medical image 712. GUI 204 can display medical image 712 and the selected landmarks 718a-c and region of interest 720 in viewport 204a, and display digital pathology image 728 in viewport 204b.

### IV. Method

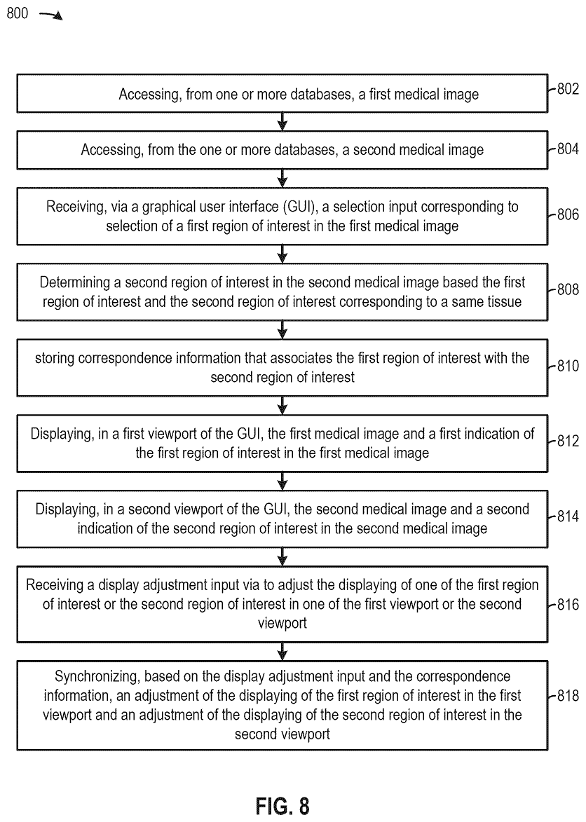

FIG. 8 illustrates a method 800 of displaying multi-modal medical images. Method 800 can be performed by multi-modal medical images correlating system 200.

Method 800 starts with step 802, in which the system accesses a first medical image from one or more databases. Moreover, in step 804, the system accesses a second medical image from the one or more databases.

Specifically, the first medical image can include a digital radiology image, such as a PET image that reveals a distribution of radioactive levels within the subject's body, whereas the second medical image can include a digital pathology image of the subject's tissue. In some example, the distribution of radioactive levels shown in the PET image can identify potential tumor locations in the subject's body, whereas digital pathology image can include an image of a sample (e.g., a tissue specimen) collected from the subject that has been stained (e.g., H&E staining, IHC staining, fluorescent tagging, etc.) and/or illuminated (e.g., fluorescent illumination, bright-field illumination, etc.) to reveal suspected tumor cell. In some examples, both the first medical image and the second medical image can include digital radiology images or digital pathology images but obtained using different techniques (e.g., different types of staining, different types of illuminations, etc.) to reveal different information. The one or more databases may include digital radiology images database 130, digital pathology images database 140, etc., and can be part of, for example, an electronic medical record (EMR) system, a picture archiving and communication system (PACS), a Digital Pathology (DP) system, a laboratory information system (LIS), and a radiology information system (RIS).

In step 806, the system receives, via a graphical user interface (GUI), a selection input corresponding to selection of a first region of interest in the first medical image.

Specifically, the system may provide a GUI, such as GUI 204. Examples of the selection input are shown in FIG. 7A-FIG. 7F. The selection input can include a selection of one or more first image locations in the first medical image as one or more first landmark points. The first region of interest can encompass the first landmark points. The first region of interest can be of various geometric shapes, such as a triangular shape, a rectangular shape, a freeform shape, etc., which can be based on the number of first landmark points. For example, the first region of interest can correspond to a region having a radioactive level in a PET image, which can indicate the presence of a tumor that metabolizes a radiolabeled glucose tracer injected into the subject's body. In some examples, the selection input can also include a direct selection of the first region of interest by a user. In some examples, an image processing application of the multi-modal medical images correlating system can process the first medical image by comparing the radioactive level revealed in the PET image with a threshold. One or more candidate first regions of interest in the first image can be defined based on the comparison result. For example, the one or more candidate first regions of interest in the first image can be defined based on regions having radioactive level higher than the threshold. Multiple candidate first regions of interest may be identified in a case where there are multiple suspected tumor sites in the subject's body. The selection input can be received from the user to select one of the candidate first regions of interest as the first region of interest that corresponds to, for example, a tumor site at a particular location of the subject's body. Based on the selection input, the system can determine various information of the first region of interest including, for example, a first location (e.g., a center location) of the first region of interest, a shape of the first region of interest, a size of the first region of interest, etc.

In step 808, the system determines a second region of interest in the second medical image based the first region of interest and the second region of interest corresponding to a same tissue.

Specifically, the second region of interest can be determined based on, for example, determining the tissue (e.g., a tumor tissue) represented by the first region of interest, followed by identifying the second region of interest in the second medical image that corresponds to the same tissue (e.g., the same tumor tissue). In some examples, the determination can be based on receiving a second selection input from the user. The second selection input may include selection of one or more second image locations in the second medical image as one or more second landmark points, and the second region of interest can encompass the second landmark points. In some examples, the information can also be determined based on inputs from the user. For example, as shown in FIG. 7A-FIG. 7F, the GUI may provide a corresponding regions of interest input option to enter landmark points of a pair of corresponding regions of interest in the first medical image and in the second medical image. By receiving the first and second landmark points via the corresponding region of interest input option, the multi-modal medical images correlating system can determine the information indicating that the first region of interest and the second region of interest correspond to the same tissue. The system can also determine various information of the second region of interest including, for example, a second location (e.g., a center location) of the second region of interest, a shape of the second region of interest, a size of the second region of interest, etc., based on the landmark points in the second medical image.

In some examples, referring to FIG. 5A-FIG. 5C, the second region of interest can also be determined by a machine learning model of the multi-modal medical images correlating system. The machine learning model can determine, for each pixel of the second medical image, a likelihood of the pixel belonging to the tissue, and classify that a pixel belongs to the tissue, and that the pixel is to be included in the second region of interest, if the likelihood exceeds a threshold. Based on the classification results, the multi-modal medical images correlating system can then determine the second region of interest in the second medical image to include pixels that are classified as part of the tissue. In some examples, the machine learning model can include a deep convolutional neural network (CNN) comprising multiple layers. The CNN can perform convolution operations between the second medical image and weight matrices representing features of the tissue to compute the likelihoods of the pixels belonging to the tissue, and to determine the pixels that are part of the second region of interest. The system can then determine various information of the second region of interest including, for example, a second location (e.g., a center location) of the second region of interest, a shape of the second region of interest, a size of the second region of interest, etc., based on pixels determined to be part of the second region of interest.

In step 810, the system stores correspondence information that associates the first region of interest with the second region of interest.

Examples of the correspondence information are shown in FIG. 3A-FIG. 3C. The correspondence information can indicate one or more first locations of the first region of interest, one or more second locations of the second region of interest, and the correspondence between the first region of interest and the second region of interest. The first and second locations can include, for example, the boundary locations, center locations, etc., of the first region of interest and the second region of interest. In some examples, the correspondence information can include the pixel locations of the first landmarks and the second landmarks that can define, respectively, the first location of the first region of interest and the second location of the second region of interest. In some examples, the correspondence information may further include additional information, such as the locations of the boundaries of the first region of interest and the second region of interest, the file names of the first medical image and the second medical image, the type of tissue represented in the regions of interest, etc. In some examples, the correspondence information may include a data structure, such as a mapping table, that maps the first region of interest to the second region of interest. In some examples, the first medical image can be part of a 3D PET image, and the mapping table can include three dimensional coordinates of the first region of interest. The mapping table can also map the electronic file names of the first medical image to the second medical image if both medical images are 2D images. In a case where the first medical image is part of a 3D PET image that comprises multiple 2D PET images obtained at different longitudinal positions, the mapping table can map first regions of interest in multiple 2D PET images to second regions of interest in multiple second medical images. Such arrangements allow the multi-modal medical images correlating system to access the mapping table and the regions of interest information after accessing the first medical image and the second medical image.

In step 812, the system displays, in a first viewport of the GUI, the first medical image and a first indication of the first region of interest in the first medical image. Moreover, in step 814, the system displays, in a second viewport of the GUI, the second medical image and a second indication of the second region of interest in the second medical image. Referring to FIG. 7A-FIG. 7F, the indications of regions of interest can be in various forms, such as the landmarks that define the region of interest, a geometric shape representing the region of interest, various forms of annotations, etc.

In step 816, the system receives a display adjustment input via to adjust the displaying of one of the first region of interest or the second region of interest in one of the first viewport or the second viewport. The display adjustment input can include, for example, a zoom-in/zoom-out input, a panning input, a rotation input, etc., to adjust the displaying of a region of interest in the viewport that receives the display adjustment input.

In step 818, the system synchronizes, based on the display adjustment input and the correspondence information, an adjustment of the displaying of the first region of interest in the first viewport and an adjustment of the displaying of the second region of interest in the second viewport.

The multi-modal medical images correlating system can perform the synchronization based on the display adjustment input and the correspondence information. As a result of the synchronization, various settings of the display, such as a degree of magnification, the portion of the region of interest selected for display, a viewpoint of the region of interest, etc., are applied to both viewports, such that both viewports can display the same region indicated by the same set of coordinates in the first and second medical images. For example, in a case where a zoom-in input is received at the first viewport to zoom into the first region of interest in the first viewport, the multi-modal medical images correlating system can compute a degree of magnification based on the zoom-in input, and magnify the first region of interest in the first viewport by the degree of magnification. In addition, the multi-modal medical images correlating system can also identify the second region of interest at the second location of the second medical image (based on the correspondence information), magnify the second region of interest by the same degree of magnification in the second viewport so that the first region of interest and the second region of interest are displayed to the same scale. As another example, a panning input is received at the first viewport to pan to a selected portion of the first region of interest, and the multi-modal medical images correlating system can display the selected portion of the first region of interest. In addition, based on a mapping between first location of the first region of interest and the second location of the second region of interest, the multi-modal medical images correlating system can determine the corresponding portion of the second region of interest, and display the corresponding portion of the second region of interest in the second viewport.

### V. Computer System

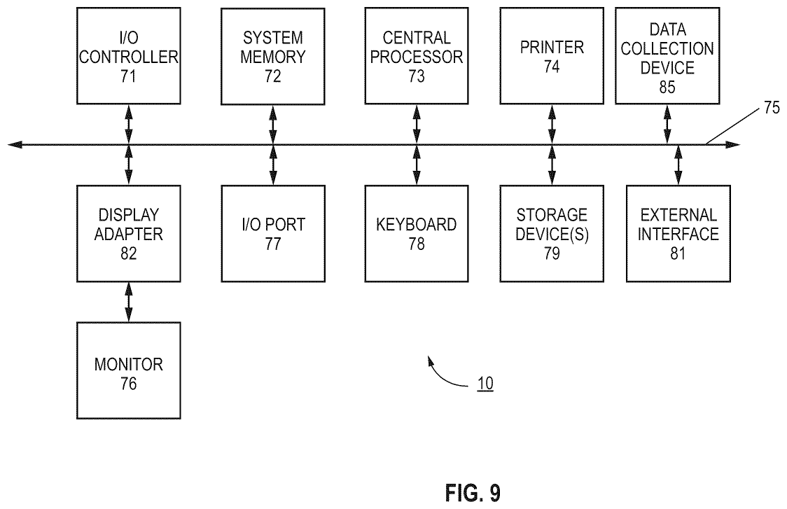

Any of the computer systems mentioned herein may utilize any suitable number of subsystems. Examples of such subsystems are shown in FIG. 9 in computer system 10 (which may include one or more cloud computers, which may facilitate one or more local deployments). In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 10 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g. Ethernet, Wi-Fi, etc.) can be used to connect computer system FIG. 10 to a wide area network such as the Internet, a mouse input device, or a scanner. I/O port 77 can receive inputs (e.g., selection of landmarks, display adjustments inputs, etc.) from a peripheral device (e.g., a computer mouse), and provide the inputs to GUI 204. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, a digital scanner for digital pathology images, an imaging scanner for radiology images, etc. Any of the data mentioned herein can be output from one component to another component and can be output to the user.

A computer system can include a plurality of the same components or subsystems, e.g., connected together by external interface 81 or by an internal interface. In some embodiments, computer systems, subsystems, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application, including multi-modal medical images correlating system 200 as its components as described in FIG. 2A-FIG. 9, may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the invention. However, other embodiments of the invention may be directed to specific embodiments relating to each individual aspect, or specific combinations of these individual aspects.

The above description of example embodiments of the invention has been presented for the purposes of illustration and description. It is not intended to be exhaustive or to limit the invention to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

What is claimed is:

1. A computer-implemented method, comprising:
accessing, from one or more databases, a first medical image;
accessing, from the one or more databases, a second medical image;
receiving, via a graphical user interface (GUI), a selection input corresponding to selection of a first region of interest in the first medical image;
determining a second region of interest in the second medical image based the first region of interest and the second region of interest corresponding to a same tissue;
storing correspondence information that associates the first region of interest with the second region of interest;
displaying, in a first viewport of the GUI, the first medical image and a first indication of the first region of interest in the first medical image;
displaying, in a second viewport of the GUI, the second medical image and a second indication of the second region of interest in the second medical image;
receiving a display adjustment input via to adjust the displaying of one of the first region of interest or the second region of interest in one of the first viewport or the second viewport; and
synchronizing, based on the display adjustment input and the correspondence information, an adjustment of the displaying of the first region of interest in the first viewport and an adjustment of the displaying of the second region of interest in the second viewport.

2. The method of claim 1, wherein the first medical image and the second medical image comprise at least one of digital radiology images, or digital pathology images.

3. The method of claim 2, wherein the first medical image comprises one or more positron emission tomography (PET) images; and
wherein the second medical image comprises a digital pathology image of a specimen slide.

4. The method of claim 3, wherein the digital pathology image captures the specimen slide processed based on one of: Hematoxylin and Eosin (H&E) staining or Immunohistochemistry (IHC) staining.

5. The method of claim 3, wherein the digital pathology image is obtained based on at least one of: a fluorescent illumination, or a bright-field illumination.

6. The method of claim 1, wherein the selection input comprises a selection of a plurality of first landmarks in the first viewport; and
wherein the method further comprises:
determining first location information of the first region of interest in the first medical image based on locations of the plurality of first landmarks in the first viewport and a display scaling factor of the first medical image in the first viewport; and
storing the first location information in the correspondence information.

7. The method of claim 6, wherein the method further comprises:
receiving a selection of a plurality of second landmarks in the second viewport;
determining second location information of the second region of interest in the second medical image based on locations of the plurality of second landmarks in the second viewport and a display scaling factor of the second medical image in the second viewport; and
storing the second location information in the correspondence information.

8. The method of claim 7, further comprising:
detecting, by the GUI, a selection of an option to select landmarks of corresponding regions of interest in the first viewport and the second viewport,
wherein the determination that the first region of interest and the second region of interest correspond to the same tissue is based on detecting the selection of the option.

9. The method of claim 1, further comprising:
performing a first image processing operation on the first medical image to determine the first region of interest;
performing a second image processing operation on the second medical image to determine the second region of interest;
receiving the selection input as a confirmation that the first region of interest determined by the first image processing operation and the second region of interest determined by the second image processing operation correspond to the same tissue; and
storing the correspondence information based on the confirmation.

10. The method of claim 9, wherein the first image processing operation comprises comparing a radioactive level represented by pixels of the first medical image against a threshold; and
wherein the first region of interest is determined based on a subset of the pixels for which the radioactive level exceeds the threshold.

11. The method of claim 10, wherein the second image processing operation comprises detecting one or more stain patterns indicative of cancer cells; and
wherein the second region of interest is determined based on the result of the detection.

12. The method of claim 11, wherein the second image processing operation is performed by a convolutional neural network.

13. The method of claim 1, further comprising:
generating, using a learning system, a prediction that the first region of interest and the second region of interest correspond to the same tissue;
displaying, in the GUI, the prediction; and
receiving the selection input as a confirmation of the prediction.

14. The method of claim 1, wherein the display adjustment input comprises at least one of: a zoom-in input, a zoom-out input, a panning input, or a rotation input.

15. The method of claim 14, wherein synchronizing an adjustment of the displaying of the first region of interest in the first viewport and an adjustment of the displaying of the second region of interest in the second viewport is performed such that the first region of interest and the second region of interest are displayed by a same degree of magnification in, respectively, the first viewport and the second viewport.

16. The method of claim 14, wherein synchronizing an adjustment of the displaying of the first region of interest in the first viewport and an adjustment of the displaying of the second region of interest in the second viewport is performed such that a same portion of the first region of interest and second region of interest are displayed in, respectively, the first viewport and the second viewport.

17. The method of claim 14, wherein synchronizing an adjustment of the displaying of the first region of interest in the first viewport and an adjustment of the displaying of the second region of interest in the second viewport is performed such that the first region of interest and second region of interest are rotated by a same degree in, respectively, the first viewport and the second viewport.

18. The method of claim 1, further comprising:
generating an analytics output based on the first region of interest and the second region of interest corresponding to the same tissue,
wherein the analytics output comprises at least one of: a prediction of a cancer diagnosis, a verification of a prior surgical procedure, a classification of the same tissue captured in the first region of interest and in the second region of interest, or a research operation regarding a treatment.

19. A computer-program product tangibly embodied in a non-transitory machine-readable storage medium, including instructions configured to cause one or more data processors to perform a set of actions including:
accessing, from one or more databases, a first medical image;
accessing, from the one or more databases, a second medical image;
receiving, via a graphical user interface (GUI), a selection input corresponding to selection of a first region of interest in the first medical image;
determining a second region of interest in the second medical image based the first region of interest and the second region of interest corresponding to a same tissue;
storing correspondence information that associates the first region of interest with the second region of interest;
displaying, in a first viewport of the GUI, the first medical image and a first indication of the first region of interest in the first medical image;
displaying, in a second viewport of the GUI, the second medical image and a second indication of the second region of interest in the second medical image;
receiving a display adjustment input via to adjust the displaying of one of the first region of interest or the second region of interest in one of the first viewport or the second viewport; and
synchronizing, based on the display adjustment input and the correspondence information, an adjustment of the displaying of the first region of interest in the first viewport and an adjustment of the displaying of the second region of interest in the second viewport.

20. A system comprising:
one or more data processors; and
a non-transitory computer readable storage medium containing instructions which, when executed on the one or more data processors, cause the one or more data processors to perform a set of actions including:
accessing, from one or more databases, a first medical image;
accessing, from the one or more databases, a second medical image;
receiving, via a graphical user interface (GUI), a selection input corresponding to selection of a first region of interest in the first medical image;
determining a second region of interest in the second medical image based the first region of interest and the second region of interest corresponding to a same tissue;
storing correspondence information that associates the first region of interest with the second region of interest;
displaying, in a first viewport of the GUI, the first medical image and a first indication of the first region of interest in the first medical image;
displaying, in a second viewport of the GUI, the second medical image and a second indication of the second region of interest in the second medical image;
receiving a display adjustment input via to adjust the displaying of one of the first region of interest or the second region of interest in one of the first viewport or the second viewport; and
synchronizing, based on the display adjustment input and the correspondence information, an adjustment of the displaying of the first region of interest in the first viewport and an adjustment of the displaying of the second region of interest in the second viewport.
